(19) United States
(12) Patent Application Publication
(10) Pub. No.: US 2009/0281531 A1
(43) Pub. Date: Nov. 12, 2009

(54) INTERVENTIONAL AND THERAPEUTIC ELECTROMAGNETIC ENERGY SYSTEMS

(76) Inventors: Ioana M. Rizoiu, San Clemente, CA (US); Peter Chueh, Tustin, CA (US); Manvel Artyom Andriasyan, San Diego, CA (US); Dmitri Boutoussov, Dana Point, CA (US)

Correspondence Address: Kenton R. Mullins;Stout, Uxa, Buyan & Mullins, LLP, Suite 300, 4 Venture, Irvine, CA, 92618, US

(21) Appl. No.: 12/437,485

(22) Filed: May 7, 2009

Related U.S. Application Data
(63) Continuation-in-part of application No. 11/033,032, filed on Jan. 11, 2005.
Application No. 11/033,032 is a continuation-in-part of application No. 10/993,498, filed on Nov. 18, 2004, now Pat. No. 7,108,693.
Application No. 10/993,498 is a continuation of application No. 10/164,451, filed on Jun. 6, 2002, now Pat. No. 6,821,272.
Application No. 10/164,451 is a continuation of application No. 09/883,607, filed on Jun. 18, 2001, now abandoned.
Application No. 09/883,607 is a continuation of application No. 08/903,187, filed on Jun. 12, 1997, now Pat. No. 6,288,499.
Application No. 08/903,187 is a continuation-in-part of application No. 08/522,503, filed on Aug. 31, 1995, now Pat. No. 5,741,247.
(60) Provisional application No. 61/051,315, filed on May 7, 2008.

(51) Int. Cl. A61B 18/22

(52) U.S. Cl. 606/11; 606/13; 604/20; 606/16

(57) ABSTRACT

Output optical energy pulses having relatively high energy magnitudes and short durations are combined with optical energy pulses having relatively low energy magnitudes and long durations.

### CROSS-REFERENCE TO RELATED APPLICATIONS

This application claims the benefit of U.S. Provisional Application No. 61/051,315 (BI8108PR), filed May 7, 2008 and entitled INTERVENTIONAL AND THERAPUTIC ELECTROMAGNETIC ENERGY SYSTEMS, the contents of which are expressly incorporated herein by reference. This application is a continuation-in-part of U.S. application Ser. No. 11/033,032 (BI9842P), filed Jan. 10, 2005 and entitled ELECTROMAGNETIC ENERGY DISTRIBUTIONS FOR ELECTROMAGNETICALLY INDUCED DISRUPTIVE CUTTING, which claims the benefit of U.S. Provisional Application No. 60/535,004, filed Jan. 8, 2004, the contents of both which are expressly incorporated herein by reference. U.S. application Ser. No. 11/033,032 is a continuation-in part application of U.S. application Ser. No. 10/993,498, filed Nov. 18, 2004, now U.S. Pat. No. 7,108,693, which is a continuation application of U.S. application Ser. No. 10/164,451, filed Jun. 6, 2002, now U.S. Pat. No. 6,821,272, which is a continuation application of U.S. application Ser. No. 09/883,607, filed Jun. 18, 2001, which is a continuation application of U.S. application Ser. No. 08/903,187, filed Jun. 12, 1997, now U.S. Pat. No. 6,288,499, which is a continuation-in-part of U.S. application Ser. No. 08/522,503, filed Aug. 31, 1995, now U.S. Pat. No. 5,741,247, all of which are commonly assigned and the contents of which are expressly incorporated herein by reference.

### BACKGROUND OF THE INVENTION

1. Field of the Invention

The present invention relates generally to electronic devices and, more particularly, to output optical energy distributions of lasers.

2. Description of Related Art

A variety of electromagnetic laser energy generating architectures have existed in the prior art. A solid-state laser system, for example, generally comprises a laser rod for emitting coherent light and a source for stimulating the laser rod to emit the coherent light. Flashlamps are typically used as stimulation sources for middle infrared lasers between 2.5 μm and 3.5 μm, such as Er,Cr:YSGG and Er:YAG laser systems, for example. The flashlamp is driven by a flashlamp current, which comprises a predetermined pulse shape and a predetermined frequency.

The flashlamp current drives the flashlamp at the predetermined frequency, to thereby produce an output flashlamp light distribution having substantially the same frequency as the flashlamp current. This output flashlamp light distribution from the flashlamp drives the laser rod to produce coherent light at substantially the same predetermined frequency as the flashlamp current. The coherent light generated by the laser rod has an output optical energy distribution over time that generally corresponds to the pulse shape of the flashlamp current.

While flashlamps are typically used as stimulation sources for laser systems, for example, diodes may be used as well for the excitation source. The use of diodes for generating light amplification by stimulated emission is discussed in the book Solid-State Laser Engineering, Fourth Extensively Revised and Updated Edition, by Walter Koechner, published in 1996, the contents of which are expressly incorporated herein by reference.

The pulse shape of the output optical energy distribution over time typically comprises a relatively gradually rising energy that ramps up to a maximum energy, and a subsequent decreasing energy over time. The pulse shape of a typical output optical energy distribution can provide a relatively efficient operation of the laser system, which corresponds to a relatively high ratio of average output optical energy to average power inputted into the laser system.

The prior art pulse shape may be suitable for cutting procedures, for example, where the output optical energy is directed onto a target surface to induce cutting of the contact tissue. However, when thermal cutting is employed utilizing certain conventional procedures, undesirable secondary damage, such as charring or burning of surrounding structures or tissues, may occur. Newer cutting procedures, however, may not altogether rely on laser-induced thermal heating only. More particularly, a cutting mechanism, such as that disclosed in U.S. Pat. No. 5,741,247, directs output optical energy from a laser system first into a distribution of atomized fluid particles located in a volume of space above the target surface. Disruptive (e.g., mechanical, thermo-mechanical, and other) cutting forces then can be imparted onto the tissue. In certain implementations, at least a portion of the output optical energy interacts with the atomized fluid particles, causing the atomized fluid particles to expand, wherein electromagnetically-induced disruptive forces may be imparted onto the target surface. As a result of the unique interactions of the output optical energy with the atomized fluid particles, many prior art output optical energy distribution pulse shapes and frequencies have not been especially suited for providing optimal electromagnetically-induced disruptive (e.g., mechanical, thermo-mechanical, and other) processes such as for example cutting, removing, ablating, cleaning and others. Specialized output optical energy distributions may be advantageous for optimal cutting, for example, when the output optical energy is directed into a distribution of atomized fluid particles for effectuating a transfer of pulse energy that is initially coupled into the highly absorbing molecules of the atomized fluid particles and secondly into the highly absorbing molecules of the material to be cut.

### SUMMARY OF THE INVENTION

The present invention provides an apparatus having output optical energy distributions that can be tailored for delivering custom, treatment-specific tissue treatments. The output optical energy distributions disclosed herein permit a cutting apparatus to cut a target surface, such as body tissue, with reduced, and preferably no, undesirable secondary effects (e.g., damage) to the target surface. The apparatus may cut the target surface without requiring application of additional fluids, or in other words, the cutting of the target tissue may occur by thermal energy of the output energy alone, or in combination, with disruptive (e.g., mechanical, thermo-mechanical and other) energy imparted by or in connection with disruption of fluid particles located above the target surface, on the target surface, or within the target surface.

In one implementation, output optical energy from a laser system can be directed first into a distribution of atomized fluid particles located in a volume of space just above the target surface, and then into the material wherein for example absorbing molecules are exposed to very fast rising pulses with a steep slope, causing a localized expansion of that component of the material and subsequent removal of that material with, in some embodiments, minimal to no thermal heat deposition into the material.

The present invention, together with additional features and advantages thereof, may best be understood by reference to the following description taken in connection with the accompanying illustrative drawings.

### BRIEF DESCRIPTION OF THE DRAWINGS

FIGS. 1 to 3 illustrate plots of energy versus time for output optical energy waveforms, according to the present invention, that can be outputted by an electromagnetic energy output system such as a laser module;

FIG. 4 is a plot of flashlamp-driving current versus time in accordance with the present invention;

FIG. 5 is a plot of output optical energy versus time for a laser system in accordance with the present invention; and

FIGS. 6A to 6F are plots of S-mode and L-mode energy combinations.

### DETAILED DESCRIPTION OF THE INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not presumed, automatically, to be to precise scale in all embodiments. That is, they are intended to be examples of implementations of various aspects of the present invention and, according to certain but not all embodiments, to be to scale. While, according to certain implementations, the structures depicted in any one or more of these figures are to be interpreted to be to scale, in other implementations the same shapes/structures should not. In certain aspects of the invention, use of the same reference designator numbers in the drawings and the following description is intended to refer to similar or analogous, but not necessarily the same, components and elements. According to other aspects, use of the same reference designator numbers in these drawings and the following description is intended to be interpreted as referring to the same or substantially the same, and/or functionally the same, components and elements. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent accompanying this disclosure is to discuss exemplary embodiments with the following detailed description being construed to cover all modifications, alternatives, and equivalents of the embodiments as may fall within the spirit and scope of the invention as defined by the appended claims. It is to be understood and appreciated that the process steps and structures described herein do not cover a complete process flow for the manufacture of the disclosed structures.

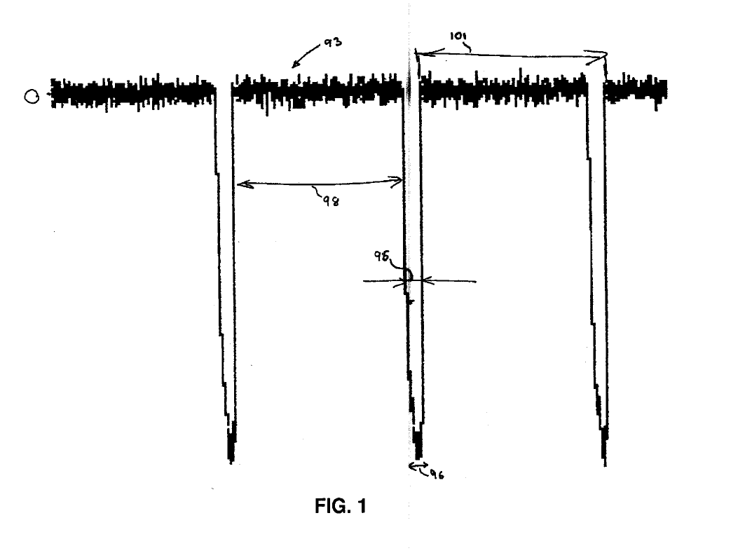

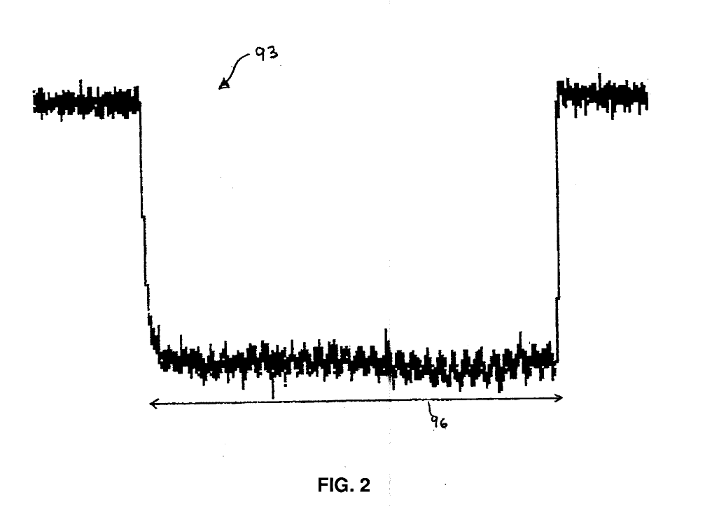

Referring more particularly to the drawings, FIG. 1 illustrates a plot of energy versus time for an output optical energy waveform 93, according to an aspect of the present invention, that can be outputted by an electromagnetic energy output system. FIG. 2 is a magnified view of the plot of energy versus time for the output optical energy waveform 93 of FIG. 1.

Each of the pulses of the output optical energy waveform 93 comprises a plurality of micropulses. The micropulses correspond to population inversions within the laser rod as coherent light is generated by stimulated emission. Particles, such as electrons, associated with impurities of the laser rod absorb energy from the impinging incoherent radiation and rise to higher valence states. The particles that rise to metastable levels remain at this level for periods of time until, for example, energy particles of the radiation excite stimulated transitions. The stimulation of a particle in the metastable level by an energy particle results in both of the particles decaying to a ground state and an emission of twin coherent photons (particles of energy). The twin coherent photons can resonate through the laser rod between mirrors at opposing ends of the laser rod, and can stimulate other particles on the metastable level, to thereby generate subsequent twin coherent photon emissions. This process is referred to as light amplification by stimulated emission. With this process, a twin pair of coherent photons will contact two particles on the metastable level, to thereby yield four coherent photons. Subsequently, the four coherent photons will collide with other particles on the metastable level to thereby yield eight coherent photons.

The amplification effect will continue until a majority of particles, which were raised to the metastable level by the stimulating incoherent light from the diode, have decayed back to the ground state. The decay of a majority of particles from the metastable state to the ground state results in the generation of a large number of photons, corresponding to an upwardly rising micropulse. As the particles on the ground level are again stimulated back up to the metastable state, the number of photons being emitted decreases, corresponding to a downward slope in the micropulse. The micropulse continues to decline, corresponding to a decrease in the emission of coherent photons by the laser system. The number of particles stimulated to the metastable level increases to an amount where the stimulated emissions occur at a level sufficient to increase the number of coherent photons generated. As the generation of coherent photons increases, and particles on the metastable level decay, the number of coherent photons increases, corresponding to an upwardly rising micropulse.

The output optical energy waveform 93 according to an aspect of the invention comprises first-characteristic electromagnetic energy generated, for example, by a diode laser to have a wavelength, pulse, and power density suitable for imparting first effects, such as coagulating or performing therapy on, for example, soft tissue. The first characteristic electromagnetic energy, according to one embodiment, can be referred to as L-mode (relatively low peak power compared to the second-characteristic electromagnetic energy) electromagnetic energy or L-mode (relatively long pulse width compared to the second-characteristic electromagnetic energy) electromagnetic energy. In the case of a diode laser, the diode light pump or the at least one diode can comprise a diode array, and the diode or diode array can be optically aligned to side pump the gain medium. In one implementation, the diode light pump can be placed, for example, within an optical cavity so that the diode or diode array is optically aligned to side pump the gain medium. According to one aspect, the first effects accomplish one or more of low-level laser or light therapy (LLLT), photo dynamic therapy (PDT) or biostimulation of one or more tissues to rejuvenate, treat, or otherwise impart energy into tissues. Low-level therapeutic optical energy applications are described in co-pending U.S. application Ser. No. 11/447,605, filed Jun. 5, 2006 and entitled TISSUE TREATMENT DEVICE AND METHOD, the entire contents of which are expressly incorporated herein by reference.

With continuing reference to FIGS. 1 and 2, the output optical energy waveform 93 (L-mode) according to an aspect of the invention is generated by a laser (e.g., a diode laser) to have a wavelength of 940 nanometers, and can be delivered, for example, in a CW (continuous wave) or a QCW (quasi-continuous wave) mode of operation. Use of the term “L-mode” is intended merely to be an abbreviated label for the term “first-characteristic electromagnetic energy” and is not intended to attribute by its name any characteristic with regard to the power or pulse width.

The gain medium can comprise an active heterostructure and substrate of, for example, AlGa(In)As/GaAs, wherein the Ga of the active heterostructure can be substituted for and/or combined with In. Another exemplary implementation can comprise AlGaInP(As)/GaAs, wherein the P of the active heterostructure can be substituted for and/or combined. Exemplary implementations may include, for example, AlGaAs, InGaAs, GaAs and InP lasers.

As presently embodied, the output optical energy waveform 93 is delivered in a pulsed-format mode of operation that is highly repetitive in time and intensity to provide, for example, relatively precise and predictable cutting. As compared, for example, to a wavelength of 810 nanometers, with other things being equal, the wavelength of 940 nanometers has been determined by the present inventors to have an absorption that is about two times greater for hemoglobin (for enhanced homeostasis) and about 20% greater for oxyhemoglobin. Alternative wavelengths which can be used according to modified aspects of the present invention can be, for example, 915 nanometers, 960 nanometers and 980 nanometers. Other alternative wavelengths which can be used in other modified aspects of the invention can comprise the mentioned wavelengths, plus or minus about 50 nanometers. In certain embodiments, the wavelengths can be, for example, within a range of about 400 nanometers to 1500 nanometers.

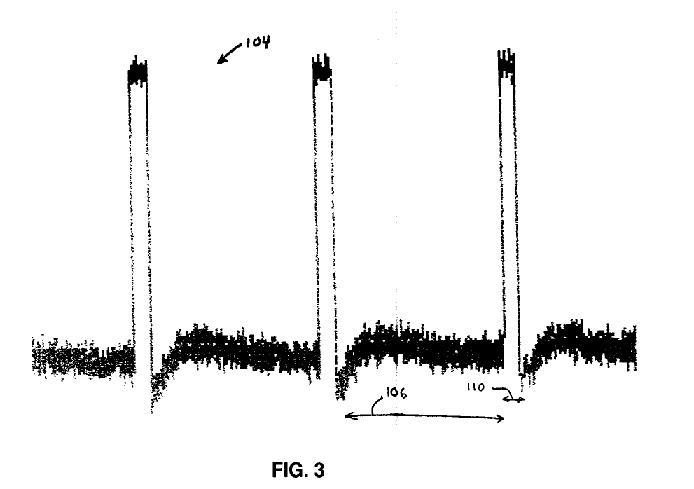

As shown in FIG. 1, each pulse of the output optical energy waveform 93 can comprise, for example, a pulse duration 96 of about 50 microseconds, a pulse interval 98 of about 450 microseconds, and a pulse period of about 500 microseconds. The magnified view of a pulse featured in FIG. 2 shows that the pulse duration 96 has room for being further reduced in duration. For example, the pulse duration 96 can, according to certain embodiments, be reduced from about 50 microseconds all of the way down to about 10 microseconds. Thus, as illustrated, the output optical energy waveform 93 can comprise a repetition rate of about 2 kHz. The repetition rate can also be, for example, about 10 kHz, corresponding to a pulse period of about 100 microseconds. In certain embodiments, the repetition rate can be, for example, from about 1 Hz to 100 Hz. The full-width half-max of the pulse may be about 50 to 100 microseconds. The depicted output optical energy waveform 93 thus has a pulse duration 96 and a pulse interval 98 which are both on the order of microseconds. The pulse period is indicated with reference designator number 101 in the depiction of FIG. 1. FIG. 3 shows an output optical energy waveform 104 comprising, for example, a pulse duration 106 of about 500 microseconds and a pulse interval 110 of about 50 microseconds. In certain embodiments, the pulse duration can be greater, such as within a range of about 50 microseconds to 3,000 microseconds.

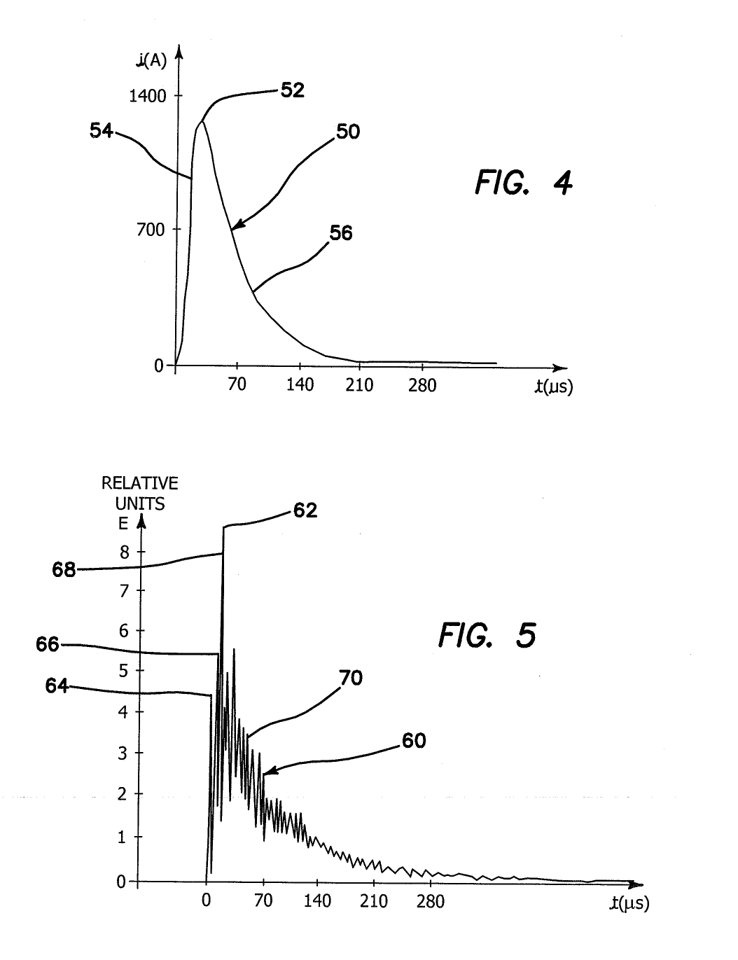

FIG. 4 illustrates the flashlamp driving current 50 of one implementation of an aspect of the present invention, which passes from an inductor to a flashlamp of a flashlamp driving circuit (not shown). The flashlamp driving current can have a pulse width greater than about 0.25 microseconds and, in some implementations, in a range of 50 to 300 microseconds. In certain embodiments, the pulse width can be from about 10 to about 300 microseconds, and the repetition rate can be, for example, from about 1 Hz to about 100 Hz. In the illustrated embodiment, the pulse width is about 200 microseconds. The flashlamp driving current 50 comprises a maximum value 52, an initial ramp portion 54, and a declining current portion 56. The flashlamp (not shown) can comprise a cylindrical glass tube having an anode, a cathode, and a gas there between such as Xenon or Krypton. An ionizer circuit (not shown) ionizes the gas within the flashlamp. As the flashlamp-driving current is applied to the anode of the flashlamp, the potential between the anode and the cathode increases. This potential increases as the flashlamp-driving current increases, as indicated by the initial ramp 54. Current flows through the gas of the flashlamp, resulting in the flashlamp emitting bright incoherent light.

The flashlamp can be close-coupled to, for example, a laser rod (not shown), which preferably comprises a cylindrical crystal. The flashlamp and the laser rod are positioned parallel to one another with preferably less than 1 centimeter distance therebetween. The laser rod is suspended on two plates, and is not electrically connected to the flashlamp-driving current circuit. Although the flashlamp comprises the disclosed means of stimulating the laser rod, other means are also contemplated by the present invention. Diodes, for example, may be used instead of flashlamps for the excitation source. The use of diodes for generating light amplification by stimulated emission is discussed above with reference to FIGS. 1-3 and is also disclosed in the above-referenced book entitled Solid-State Laser Engineering.

The incoherent light from the presently preferred flashlamp impinges on the outer surface of the laser rod. As the incoherent light penetrates into the laser rod, atoms or ions within the laser rod absorb the penetrating light and subsequently emit coherent light through stimulation emission processes. The atoms or ions may comprise erbium and chromium, and the laser rod itself may comprise a crystal such as YSGG, for example. One preferred laser system comprises either an Er, Cr:YSGG solid state laser, which generates electromagnetic energy having a wavelength in a range of 2.70 to 2.80 microns, or an erbium, yttrium, aluminum garnet (Er:YAG) solid state laser, which generates electromagnetic energy having a wavelength of 2.940 microns. As presently preferred, the Er, Cr:YSGG solid state laser has a wavelength of approximately 2.789 microns and the Er:YAG solid state laser has a wavelength of approximately 2.940 microns. According to one alternative embodiment, the laser rod may comprise a YAG crystal, and the impurities may comprise erbium impurities. A variety of other possibilities exist, a few of which are set forth in the above-mentioned book Solid-State Laser Engineering. Other possible laser systems include an erbium, yttrium, scandium, gallium garnet (Er:YSGG) solid state laser, which generates electromagnetic energy having a wavelength in a range of 2.70 to 2.80 microns; an erbium, yttrium, aluminum garnet (Er:YAG) solid state laser, which generates electromagnetic energy having a wavelength of 2.94 microns; chromium, thulium, erbium, yttrium, aluminum garnet (CTE:YAG) solid state laser, which generates electromagnetic energy having a wavelength of 2.69 microns; erbium, yttrium orthoaluminate (Er:YAL03) solid state laser, which generates electromagnetic energy having a wavelength in a range of 2.71 to 2.86 microns; holmium, yttrium, aluminum garnet (Ho:YAG) solid state laser, which generates electromagnetic energy having a wavelength of 2.10 microns; quadrupled neodymium, yttrium, aluminum garnet (quadrupled Nd:YAG) solid state laser, which generates electromagnetic energy having a wavelength of 266 nanometers; argon fluoride (ArF) excimer laser, which generates electromagnetic energy having a wavelength of 193 nanometers; xenon chloride (XeCl) excimer laser, which generates electromagnetic energy having a wavelength of 308 nanometers; krypton fluoride (KrF) excimer laser, which generates electromagnetic energy having a wavelength of 248 nanometers; and carbon dioxide (CO2), which generates electromagnetic energy having a wavelength in a range of 9 to 11 microns. In certain embodiments, the wavelengths can be, for example, within a range of about 2000 nanometers to 4000 nanometers.

The output optical energy distribution over time of the laser system is illustrated in FIG. 5 at 60. The output optical energy distribution 60 according to an aspect of the invention comprises second-characteristic electromagnetic energy generated, for example, by a flashlamp or diode laser to have a wavelength, pulse, and power density suitable for imparting second effects, such as cutting on, for example, soft tissue. The second characteristic electromagnetic energy, according to one embodiment, can be referred to as H-mode (relatively high peak power compared to the first-characteristic electromagnetic energy) electromagnetic energy or S-mode (relatively short pulse width compared to the first-characteristic electromagnetic energy) electromagnetic energy. The output optical energy distribution 60 (S-mode) of the present invention can have a pulse width that is greater than about 0.25 microseconds, such as in a range of about 10 to 300 microseconds. In the illustrated embodiment, the pulse width can be about 200 microseconds. The output optical energy distribution 60 comprises a maximum micropulse value 62, a number of leading micropulses 64, 66, 68, and a portion of generally declining optical energy 70. Use of the term “S-mode” is intended merely to be an abbreviated label for the term “second-characteristic electromagnetic energy” and is not intended to attribute by name any characteristic with regard to the power or pulse width.

According to the present invention, the output optical energy distribution 60 comprises a large magnitude. This large magnitude corresponds to one or more sharply-rising micropulses at the leading edge of the pulse. As illustrated in FIG. 5, the micropulse 68 comprises a maximum value 62 which is at or near the very beginning of the pulse. Additionally, the full-width half-max value of the output optical energy distribution in FIG. 5 is approximately 70 microseconds, and in other embodiments can be between about 40 and about 65 microseconds. Applicants' invention contemplates pulses comprising full-width half-max values greater than 0.025 microseconds, such as those ranging from 10 to 150 microseconds, but other ranges may also be possible.

Another aspect of this invention is the combination of pulses from a pulse width range between 0.25 and 300 microseconds with pulses from a pulse width range between 300 microseconds and 800 microseconds.

Further, a frequency of 1 to 20 Hz is presently preferred. Alternatively, frequencies of 20-150 Hz may be used. Applicants' invention generally contemplates frequencies between 1 and 150 Hz. In case of diode-pumped, Q-switched lasers, the frequencies may be as high as in the KHz range.

As mentioned above, the full-width half-max range is defined from a beginning time, where the amplitude first rises above one-half the peak amplitude, to an ending time, where the amplitude falls below one-half the peak amplitude a final time during the pulse width. The full-width half-max value is defined as the difference between the beginning time and the ending time.

The location of the full-width half-max range along the time axis, relative to the pulse width, is closer to the beginning of the pulse than the end of the pulse. The location of the full-width half-max range is preferably within the first half of the pulse and, more preferably, is within about the first third of the pulse along the time axis. Other locations of the full-width half-max range are also possible in accordance with the present invention. The pulse rise time preferably occurs within the first 5 to 35 microseconds and, more preferably, occurs within the first 12.5 microseconds from the beginning of the pulse. The beginning time is preferably achieved within the first tenth of the pulse width.

Another feature of the output optical energy distribution 70 is that the micropulses 64, 66, 68, for example, comprise approximately one-third of the maximum amplitude 62. More preferably, the leading micropulses 64, 66, 68 comprise an amplitude of approximately one-half of the maximum amplitude 62. The slope of the output optical energy distribution 60 is greater than or equal to 5 and in another embodiment is greater than about 10. In the illustrated embodiment, the slope is about 50. In contrast, the slope of the output optical energy distribution 20 of the prior art is about 4.

In a further embodiment of the invention, such as embodiments in which the energy is used for treating and/or to improve cutting of soft tissues, the slope of the pulse may be less steep. For instance, the shape of the pulse may be smoother than the shapes discussed above. By utilizing pulses with less steep initial slopes it may be possible to achieve for example enhanced coagulation of the cut tissue.

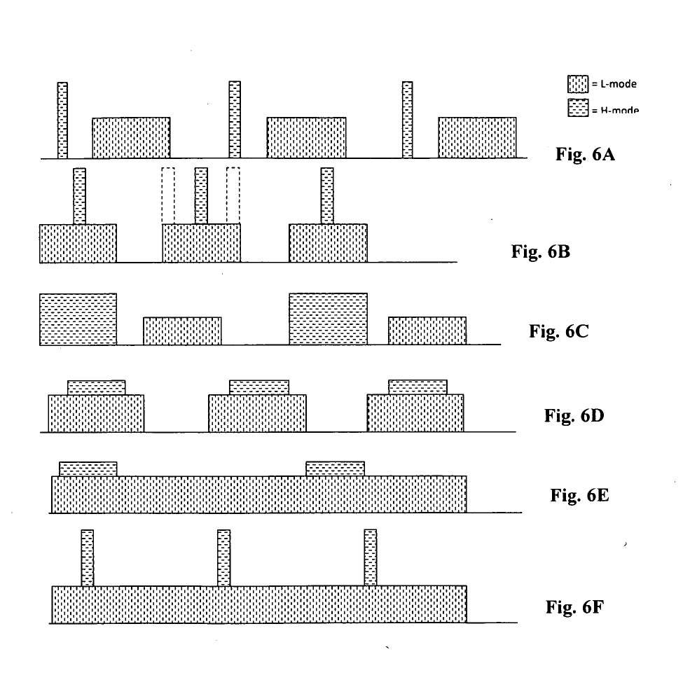

With reference to FIGS. 6A-6F, plots (x axis=time, y axis=power) of S-mode and L-mode pulse combinations are depicted according to exemplary implementations. In certain embodiments, either the S-mode or the L-mode pulses may be applied to the target alone, and in other embodiments they may be applied together in various combinations to provide, for example, a cutting and coagulating effect is achieved by alternating and/or overlapping S-mode and L-mode pulses, such as shown. In the illustrations, the S-mode pulses can comprise relatively large amplitudes compared to the L-mode pulses. For example, a cutting effect with, for example, coagulation may be obtained by providing a train of pulses in various sequences of S-mode and L-mode pulses. In one embodiment, the train of pulses may include alternating S-mode and L-mode pulses. In another embodiment, the train of pulses may include a sequence of pulses such as L-mode, L-mode, S-mode, or S-mode, S-mode, L-mode. Additional patterns or sequences may also be utilized. By utilizing alternating or changing pulse shapes, it may be possible to obtain combined effects not achievable by any single pulse shape. For example, by utilizing an S-mode pulse and L-mode pulse combination, it may be possible to create a deep cut with a relatively strong coagulation. Typically, S-mode pulses may tend to create a relatively deep cut with moderate coagulation, and L-mode pulses may tend to create a relatively shallow cut with strong coagulation. By generating pulses of different maximum amplitudes and durations, with, in one example, substantially equal energies according to an illustrated embodiment, it may be possible to obtain improvements in cutting and/or coagulating of target materials relative to systems which utilize only a single pulse type.

Typically, the L-mode pulses can have average powers from about 0.01 to 100 W, peak powers from about 1 to 100 W, pulse widths from about 1 microseconds all the way up to CW (e.g., from about 50 to 3000 microseconds), and wavelengths from about 0.4 to 1.5 microns. Typical S-mode pulses can have average powers from about 0.1 to 50 W, peak powers from about 1 k to 10k W, pulse widths from about 10 to 300 microseconds, and wavelengths from about 2 to 4 microns. A peak power value of 5 k W may correspond, for example, to about 300 mJ/pulse. Frequencies of the L-mode and S-mode pulses may range from about 1 to 100 Hz. The plot of FIG. 6A may correspond to the listed values, with a time (T) from the beginning of the S-mode pulse to the beginning of the L-mode pulse ranging from about 0 to 100 microseconds. The plot of FIG. 6B may also correspond to the listed values, with the (T) being much smaller as a consequence of the S-mode L-mode pulses overlapping (e.g., the S- and L-mode pulses may begin at the same time as represented by the left-most phantom S-mode pulse, may begin at times corresponding to the S-mode pulse being positioned in the middle of the L-mode pulse as shown, or the S- and L-mode pulses may end at the same time as represented by the right-most phantom S-mode pulse). The plot of FIG. 6C may also correspond to the listed values, with the S-mode pulses having peak powers from about 0.1 k to 1 k W and pulse widths from about 300 to 1000 microseconds, with the longer S-mode pulse having some coagulative effects (e.g., a depth of coagulation of 80-100 um) on its own and with the depth of coagulation being augmented even more with the L-mode pulses. In FIG. 6D, the S- and L-mode pulses overlap as in FIG. 6B and the S-mode pulses are relatively long as in FIG. 6C. In FIG. 6E, the S- and L-mode energies overlap as in FIG. 6B and the L-mode energy is a CW emission. FIG. 6F, shows S-mode pulse shapes as in FIGS. 6A and 6B combined with an L-mode energy in the form of a CW emission.

The output optical energy distribution 60 of the present invention may be useful for maximizing a cutting effect of an electromagnetic energy source, such as a laser, directed toward a target surface. The cutting and/or ablating effects may occur on or at the target surface, within the target surface, and/or above the target surface. Using the optical energy distributions disclosed herein, it is possible to disrupt a target surface by directing electromagnetic energy toward the target surface so that, for example, a portion of the energy is absorbed by fluid. The fluid absorbing the energy may be on the target surface, within the target surface, above the target surface, or a combination thereof. In one embodiment, the fluid absorbing the energy may comprise water and/or may comprise hydroxyl. When the fluid comprises hydroxyl and/or water which highly absorb the electromagnetic energy, these fluid molecules may begin to vibrate. As the molecules vibrate, localized heat is produced that causes expansion leading to disruption (e.g., mechanical, thermo-mechanical, or other types of mechanisms). Other types of disruption effects may occur by the absorption of the impinging electromagnetic energy by other molecules of the target surface. Accordingly, the cutting effects mediated by the energy absorption may be due to thermal properties (e.g., thermal cutting) or thermo-mechanical effects and also by absorptions of the energy by molecules (e.g., water above the target surface) that do not significantly heat the target surface. The use of the electromagnetic energy distributions disclosed herein can reduce secondary damage to the target surface, such as charring or burning, in certain embodiments wherein cutting is performed in combination with a fluid output and also in certain embodiments that do not use a fluid output. Thus, a portion of the cutting effects caused by the electromagnetic energy may be due to thermal energy, and a portion of the cutting effects may be due to disruptive (e.g., mechanical, thermo-mechanical, or other types of effects) forces generated by the disruption of molecules absorbing the electromagnetic energy.

Apparatus used to impart disruptive forces onto a target surface, and/or cut or coagulate a target surface, are structured to direct electromagnetic energy toward the target surface so that at least a portion of the energy is absorbed by fluid. One apparatus for imparting disruptive forces onto a target surface is disclosed in U.S. Pat. No. 5,741,247 entitled ATOMIZED FLUID PARTICLES FOR ELECTROMAGNETICALLY INDUCED CUTTING. The apparatus can comprise one or more of a first (e.g., trunk) fiberoptic guide (e.g., tube) and a second (e.g., output) fiberoptic guide (e.g., tube). In embodiments comprising both, the apparatus can include a housing having a first (e.g., lower) portion, a second (e.g., upper) portion, and an interfacing portion which may contain an optional focusing optic between the two guides. The first guide can be surrounded at its output (e.g., upper) end by a first abutting member (e.g., a metal cylindrical object or ferrule), and the second guide can be surrounded at its input (e.g., proximal) end by a second abutting member (e.g., a metal cylindrical object or ferrule). Either or both of the guides may be formed of calcium fluoride (CaF), calcium oxide (CaO2), zirconium oxide (ZrO2), zirconium fluoride (ZrF), sapphire, hollow waveguide, liquid core, TeX glass, quartz silica, germanium sulfide, arsenic sulfide, and germanium oxide (GeO2). Not only can the cutting effects of the apparatus be mediated by atomized fluid particles above the target surface, but the cutting effects may alternatively or additionally be mediated by the absorption of energy by fluid on or within the target surface. In one embodiment of the apparatus, the cutting effects are mediated by effects of energy absorption by a combination of fluid located above the target surface, fluid located on the target surface, or fluid located in the target surface. In one embodiment, about one-third of the impinging electromagnetic energy passes through the fluid particles and impinges onto the target surface, and a portion of that impinging energy can operate to cut or contribute to the cutting of the target surface.

The fluid can comprise water. The other fluid outputs, energy sources, and other structures and methods disclosed herein, may comprise any of the fluid outputs and other structures/methods described in U.S. Pat. No. 6,231,567, entitled MATERIAL REMOVER AND METHOD, the entire contents of which are incorporated herein by reference to the extent compatible and not mutually exclusive.

The high-intensity leading micropulses 64, 66, and 68 may impart some high peak amounts of energy that are directed toward a target surface. The energy is directed toward the target surface to obtain the desired cutting effects. For example, the energy may be directed into atomized fluid particles and the fluid and/or OH molecules present on or in the material of the target surface which in some instances can comprise water or other bio-compatible fluids, to thereby expand the fluid and induce disruptive (e.g., mechanical) cutting forces to or a disruption (e.g., mechanical disruption, thermo-mechanical or any other types) of the target surface. The trailing micropulses after the maximum micropulse 68 have been found to further help with removal or material. According to the present invention, a single large leading micropulse 68 may be generated or, alternatively, two or more large leading micropulses 68 (or 64, 66, for example) may be generated. In accordance with one aspect of the present invention, relatively steeper slopes of the pulse and S-mode pulses may lower the amount of residual heat produced in the material.

The output optical energy distributions of the present invention can be adapted for cutting, shaping, removing, and coagulating tissues and materials, and further can be adapted for imparting electromagnetic energy into atomized fluid particles over a target surface, or other fluid particles located on or within the target surface. The cutting and/or treatment effect obtained by the output optical energy distributions of the present invention can be both clean and precise and, additionally, can impart consistent cuts or other effects or disruptive forces onto target surfaces.

The apparatuses disclosed herein may be used to impart cutting forces onto biological and non-biological targets. In most embodiments, the pulse or pulses may be used to generate forces effective to cut and coagulate body tissues, such as tooth, bone or cartilage. By utilizing trains of varying pulse shapes, as described above, it may be possible to obtain improved or different cutting performances. For example, it may be possible to cut a dental surface with the S-mode, high intensity pulses, and promote closing of dental tubules by a “melting” effect associated with the longer, lower intensity pulses. Such effects may be particularly useful with for example root canal procedures, or for erosion of a tooth or teeth at the gingiva to treat desensitization, such as by closing or melting of tubules to treat desensitization. Alternatively, or in addition, the trains of pulses can be used to fuse re-model dental enamel or dentin, such as to reduce or inhibit cavities. By providing trains of L-mode and S-mode pulses as described herein, it may also be possible to cut and coagulate deep tissues, such as vascular tissues.

According to another embodiment of the present invention, a fluence of electromagnetic energy (e.g., pulses of electromagnetic energy) directed toward a target (e.g., tissue) may be employed to achieve remodeling of the target as described in U.S. application Ser. No. 11/033,032.

As an example, simultaneous emission of short (or ultrashort) and long pulses may be implemented. According to another embodiment, pulses may alternate with one type followed by another type, e.g., long and short pulses. Still, other embodiments may alternate trains of pulses wherein a first number of pulses of one type alternates with a second number of pulses of another type.

Fluence settings may range from about 0.1 J/cm² to about 25 J/cm² in an exemplary embodiment. In another embodiment, the fluence settings may range from about 0.1 J/cm² to about 10 J/cm². In yet another embodiment, the fluence settings may range from about 0.1 J/cm² to about 5 J/cm². A spot size of about 50 μm to about 1500 μm may be employed in examples of the embodiments.

Although an exemplary embodiment of the invention has been shown and described, many other changes, modifications and substitutions, in addition to those set forth in the above paragraphs, may be made by one having ordinary skill in the art without necessarily departing from the spirit and scope of this invention. For example, the methods herein disclosed may be used in the treatment of tooth or bone. Any feature or combination of features described herein are included within the scope of the present invention provided that the features included in any such combination are not mutually inconsistent as will be apparent from the context, this specification, and the knowledge of one of ordinary skill in the art.

What is claimed is:

1. An apparatus for imparting disruptive forces onto a target surface, comprising:
(a) an electromagnetic energy source configured to direct electromagnetic energy toward a target surface to impart disruptive forces onto the target surface; and
(b) a flashlamp current generating circuit that generates at least one current pulse to drive the electromagnetic energy source, the current pulses having full-width half-max range positioned substantially within a first half of the current pulse and being shaped to generate electromagnetic energy from the electromagnetic energy source that disrupts the target surface using energy that is absorbed by fluid.

2. The apparatus of claim 1, wherein:
the apparatus is constructed to place fluid on the target surface; and
electromagnetic energy generated by the current pulse is at least partially absorbed by the fluid on the target surface.

3. The apparatus of claim 2, wherein the electromagnetic energy generated by the current pulse is at least partially absorbed by fluid located within the target surface.

4. The apparatus of claim 3, wherein:
the apparatus is constructed to place fluid above the target surface; and
the electromagnetic energy generated by the current pulse is at least partially absorbed by the fluid located above the target surface.

5. The apparatus of claim 4, wherein:
the apparatus is constructed to place the fluid above the target surface as atomized fluid particles; and
electromagnetic energy generated by the current pulse is substantially absorbed by the fluid located above the target surface to impart disruptive forces onto the target surface.

6. The apparatus of claim 3, wherein at least some of the fluid within the target surface that absorbs the electromagnetic energy is not supplied from the apparatus.

7. The apparatus of claim 6, wherein:
the target surface comprises hard or soft tissue; and
the fluid within the target surface comprises water.

8. The apparatus of claim 7, wherein:
the apparatus is constructed to place fluid above the target surface; and
the electromagnetic energy generated by the current pulse is at least partially absorbed by the fluid located above the target surface.

9. The apparatus of claim 8, wherein:
the apparatus is constructed to place the fluid above the target surface as atomized fluid particles; and
electromagnetic energy generated by the current pulse is substantially absorbed by the fluid located above the target surface to impart disruptive forces onto the target surface.

10. The apparatus of claim 1, wherein the electromagnetic energy generated by the current pulse is at least partially absorbed by fluid located within the target surface.

11. The apparatus of claim 3, wherein at least some of the fluid within the target surface that absorbs the electromagnetic energy is not supplied from the apparatus.

12. The apparatus of claim 6, wherein:
the target surface comprises hard or soft tissue; and
the fluid within the target surface comprises water.

13. The apparatus of claim 12, wherein:
the apparatus is constructed to place fluid above the target surface; and
the electromagnetic energy generated by the current pulse is at least partially absorbed by the fluid located above the target surface.

14. The apparatus of claim 1, wherein the electromagnetic energy generated by the current pulse is at least partially absorbed by fluid located above the target surface.

15. The apparatus of claim 14, wherein:
the apparatus is constructed to place the fluid above the target surface as atomized fluid particles; and
electromagnetic energy generated by the current pulse is substantially absorbed by the fluid located above the target surface to impart disruptive forces onto the target surface.

16. The apparatus of claim 1, wherein the current pulse of the flashlamp current generating circuit, includes:
(i) a leading edge having a slope which is at least 5, the slope being defined on a plot of the pulse as y over x (y/x) where y is current in amps and x is time in microseconds; and
(ii) a full-width half-max value in a range from about 0.025 to about 250 microseconds.

17. The apparatus of claim 1, further comprising a fluid output that outputs fluid between an output of the electromagnetic energy source and the target surface.

18. The apparatus of claim 17, comprising a filter, which comprises fluid that is output from the fluid output, wherein the filter absorbs a portion of the energy generated by the electromagnetic energy source.

19. The apparatus of claim 18, wherein:
the fluid is atomized particles of water; and
the disruption of the target surface is caused in part by energy generated by the electromagnetic energy source other than the energy absorbed by the fluid.

20. The apparatus of claim 1, wherein the apparatus includes a fiberoptic guide which comprises germanium and which is configured to route the electromagnetic energy before it imparts the disruptive forces onto the target surface.
